(19) United States
(12) Patent Application Publication
(10) Pub. No.: US 2021/0163876 A1
(43) Pub. Date: Jun. 3, 2021

(54) PREPARATION METHOD AND SYSTEM OF LOW-CARBON JET BIOFUEL BASED ON WHOLE LIFE CYCLE

(71) Applicant: Beihang University, Beijing (CN)

(72) Inventors: Xiaoyi Yang, Beijing (CN); Ziyu Liu, Beijing (CN)

(21) Appl. No.: 15/780,183

(22) PCT Filed: Dec. 13, 2017
(86) PCT No.: PCT/CN2017/115815

§ 371 Date: May 30, 2018

(30) Foreign Application Priority Data
Sep. 11, 2017 (CN) CN201710814047.5

(51) Int. Cl. C12M 1/00; C10G 3/00; C10G 67/02; C10L 1/08; C12M 3/00; B01J 19/24

(52) U.S. Cl. CPC C12M 43/02; B01J 2219/0004; C10G 67/02; C10L 1/08; C12M 21/02; C12M 21/12; C12M 23/44; B01J 19/245; C10G 2400/08; C10L 2200/043; C10L 2270/04; C10G 2300/1014; C10L 2200/0484; C10G 2300/202; C10G 3/42

(57) ABSTRACT

Disclosed are a preparation method and a system of low-carbon jet biofuel based on whole life cycle. A low-carbon method and a system of using whole life cycle involving whole process from raw material acquisition, fuel preparation to fuel application are related. A prepared jet biofuel can be used in six types of aircrafts and engines thereof. Aircrafts using the jet biofuel can have a portion of greenhouse gas emission reduction of 50% to 80%.

### CROSS-REFERENCE TO RELATED APPLICATIONS

This application claims the priority of Chinese patent application CN 201710814047.5, entitled “Preparation method and system of low-carbon jet biofuel based on whole life cycle” and filed on Sep. 11, 2017, the entirety of which is incorporated herein by reference.

### FIELD OF THE INVENTION

The present disclosure relates to a preparation method and a system of low-carbon jet biofuel based on whole life cycle assessment, in particular, to a low-carbon method and a system of using whole life cycle involving the whole process from raw material acquisition, fuel preparation to fuel application, and belongs to the technical field of jet fuels.

### BACKGROUND OF THE INVENTION

Research and development of alternative jet fuels plays an important role in national energy safety and carbon emission reduction. Development of suitable alternative jet fuels is an important guarantee for realizing diversified aviation energy supply and ensuring national energy safety. Concerning requirements of carbon emissions made by the International Civil Aviation Organization and carbon emission reduction targets specified by the Civil Aviation Administration of China, requirements of carbon emission reduction still cannot be met only by relying on development of engine and aircraft technology, such as lightweighting and performance improvement. At present, jet biofuels have become a best practicable solution.

In a perspective of feedstock acquisition, algal biomass energy is called the “third generation” biomass energy due to many advantages thereof. It is generally believed that algae biomass energy is a most potential substitute for fossil fuels, and various countries in the world pay high attention to it. Microalgae not only absorb a large amount of carbon dioxide in a cultivation stage and do not occupy arable land, but also have advantages of rapid growth and short growth cycle. In particular, microalgae can utilize carbon dioxide emitted from coal-fired power plants and coal chemical plants as substrate for photosynthesis. Moreover, requirement of utilization can be fed back to screening of algae, the fact of which lays foundation for using algal biomass as the feedstock of jet biofuels. In a perspective of fuel preparation, microalgae have high lipid content and a high heat value. Compared with carbohydrate-based biomass, demands of hydrogen sources for hydrocarbon jet fuels are reduced. In a perspective of fuel application, since existing engines and aircrafts have at least twenty years on-service, and characterized by relatively long research & development cycles with relatively high costs in economy, microalgae jet fuels should not only meet the requirements of carbon emission reduction, but also have “drop-in” performance under a condition that structures of existing engines and aircrafts as well as handling performance thereof are not changed. That is, requirements on jet fuel properties of vaporability, fluidity, combustibility, cleanability, and safety should be ensured so as to further ensure performance and safety of aeroengines and aircrafts. To ensure the above performance, group composition and carbon number distribution of aviation kerosene are limited to a certain extent.

At present, in order to realize a jet biofuel system, the following main problems should be solved. First, a problem of sustainable supply of biological sources should be solved. Microalgae not only do not occupy arable lands, but also can meet performance requirements of raw materials for fuel preparation. Moreover, microalgae have a strong carbon dioxide fixing ability, a high growth rate, and high lipid content. Second, techniques and methods for preparation of jet biofuel have requirements of low cost, low energy consumption, and low carbon emissions. Third, low fuel consumption and low carbon emissions are required in a fuel application stage. It is required that an obtained bio-fuel not only have ready-to-use performance, i.e., structures and handling performance of existing aircrafts and engines thereof should not be changed, but also have performance and safety not less than those of petroleum-based petrol. It is important that fuel consumption, combustion efficiency and carbon emissions of the obtained bio-fuel should not be higher than those of the petroleum-based jet fuel.

Therefore, at present, it is urgent to establish a low-carbon and low energy consuming jet biofuel system.

### SUMMARY OF THE INVENTION

According to one aspect, the present disclosure provides a method for preparing a low-carbon jet biofuel based on a whole life cycle. The method comprises steps of: S1, screening and obtaining microalgae coupling with carbon spectrum characteristics of jet fuels; S2, cultivating the microalgae to obtain oleaginous microalgae, which have a strong carbon dioxide fixing ability and a high productivity of fatty acids; S3, extracting oil from the oleaginous microalgae using a flash hydrothermal method to obtain biocrude containing lipid; S4, subjecting the biocrude to a heteroatom removing process and a hydrotreating process sequentially to obtain a hydrogenated product; and S5, subjecting the hydrogenated product to a fractionation process to obtain a kerosene component and a naphtha component, the kerosene component being a jet biofuel.

According to some embodiments of the present disclosure, content of C8-C16 hydrocarbon compounds in the jet fuel is in a range from 94.5% to 98%, content of C6-C8 hydrocarbon compounds is less than 2%, and content of C17-C20 hydrocarbon compounds is less than 3.5%.

According to some specific embodiments, the hydrocarbon compounds include, but are not limited to, alkanes, cycloalkanes, aromatic hydrocarbons, and olefins.

Microalgae can transform CO₂ into high energy density glycerides. Different microalgae have fatty acid glycerides with different carbon chain lengths. Microalgae which have a fatty acid carbon chain length relatively similar to carbon spectrum characteristics of jet fuels are selected for breeding and cultivating, so that unicellular or multicellular microalgae which have components similar to carbon spectrum characteristics of jet fuels can be obtained.

According to preferred embodiments of the present disclosure, in the microalgae coupling with carbon spectrum characteristics of jet fuels, fatty acids with a carbon chain length of less than 16 make up 80% to 90% of total fatty acids of the microalgae.

The microalgae are cultivated. According to preferred embodiments of the present disclosure, strains of the oleaginous microalgae obtained by screening are cultivated preferably in a raceway pond reactor and/or a flat panel photobioreactor.

During cultivation, microalgae which have a high growth rate and a strong lipid accumulation capacity are selected to obtain the oleaginous microalgae, which have a strong carbon dioxide fixing ability and a high yield of fatty acids.

According to preferred embodiments of the present disclosure, the oleaginous microalgae have a carbon dioxide fixing ability in a range from 35 to 60 g/m²·d and a productivity of fatty acid in a range from 5 to 20 g/m²·d.

According to preferred embodiments of the present disclosure, the oleaginous microalgae have oil mass content in a range from 20% to 65% and a growth rate in a range from 20 to 30 g/m²·d.

The oleaginous microalgae of the present disclosure not only have a characteristic of coupling with carbon spectrum characteristics of jet fuels required for aircrafts, but also have other characteristics, such as high carbon dioxide fixing ability, high fatty acid yield, and fast growth rate. The oleaginous microalgae with the above characteristics have a relatively high yield of oil, and more oil per unit time can be produced. That is, time required for producing unit oil is shorter, and energy consumption is less. Therefore, low energy consumption of the microalgae during a cultivation stage is guaranteed, and thus energy consumption and greenhouse gas emissions during an entire jet fuel preparation process are also reduced. Hence, the purpose of low-carbon is achieved.

According to some specific embodiments of the present disclosure, the unicellular microalgae comprise nannochloropsis, eustigmatophyceae, and scenedesmus. The nannochloropsis have lipid content in a range from 20% to 44% and a growth rate in a range from 20 to 30 g/m²·d. Carbon spectrums of the nannochloropsis are characterized by C14, C16, and C18. In the nannochloropsis, the fatty acids containing 16 carbons are the main fatty acids, and the fatty acids containing 18 and 14 carbons are in the second rich. The multicellular algae are filamentous algae, which have lipid content in a range from 30% to 65% and a growth rate in a range from 20 to 30 g/m²·d. Carbon spectrums of the filamentous algae are characterized by C14, C16, and C18. In the filamentous algae, the fatty acids containing 16 carbons are the main fatty acids, and the fatty acids containing 18 and 14 carbons are in the second place. The above oleaginous microalgae grow fast, are suitable for stable scale cultivation, and are rich in biologically active components. Besides, the multicellular algae are easy to harvest.

According to preferred embodiments of the present disclosure, for unicellular microalgae, after cultivation is finished, preferably, pH adjustment in-situ flocculation harvest technology is used first, and then the oleaginous microalgae are harvested by using a centrifugation dewatering process. Compared with a method of adding flocculant or purely using centrifugation, energy consumption can be reduced and recycling of a culture solution is ensured by using the pH adjustment in-situ flocculation harvest technology.

According to preferred embodiments of the present disclosure, the step S3 comprises extracting lipid from the oleaginous microalgae by using the flash hydrothermal method to obtain an oil phase, a water phase, a gas phase, and algal residues, wherein the lipid enters the oil phase to form the biocrude.

The term “flash hydrothermal reaction” refers to a process in which biomass is transferred into bio-oils after a series of physical and chemical reactions in a water solution at high temperature and high pressure. Since microalgae grow in a water environment, the harvested microalgae contain a large quantity of water. For preparation of bio-fuels using an existing pyrolysis process, the microalgae obtained need to be pre-dried, which would cause great energy consumption. For a solvent extraction process, a large number of solvents are needed, and volatilization of the solvents would also result in energy consumption. The present disclosure has a significant energy saving advantage on dealing with microalgae containing water and has a significant energy saving effect by using the flash hydrothermal method. Moreover, the yield can be increased by 5% to 10% by using the flash hydrothermal method.

In a preferred embodiment of the present disclosure, the flash hydrothermal method is performed preferably in a nitrogen atmosphere at a temperature in a range from 250° C. to 30° C. with a retention time in a range from 1 s to 5 s. A relatively great yield of biocrude would be obtained with such a relatively high temperature and relatively short retention time, and a lipid recovery rate can be more than 98%.

According to some embodiments of the present disclosure, the step S3 specifically comprises treating the oleaginous microalgae in a hydrothermal reactor by using the flash hydrothermal method, followed by obtaining the oil phase, the water phase, the gas phase, and the algal residues by using a flash fractionation process, wherein the lipid enters the oil phase to form the biocrude.

The temperature, pH value, and retention time of the flash hydrothermal method can be adjusted according to mass ratios of oil, protein, and carbohydrate in the microalgae. The nitrogen content in the oil phase may be reduced by 1% to 5% so as to reduce hydrogen consumption in the hydrorefining process.

According to some embodiments of the present disclosure, the step S4 comprises removing heteroatoms from the biocrude in presence of a catalyst for removing heteroatoms to obtain a heteroatom-removed product, and then hydrotreating the heteroatom-removed product in presence of a hydrogenation catalyst to obtain a hydrogenated product.

According to preferred embodiments of the present disclosure, in the heteroatom removing process, oxygen, nitrogen and metals are removed with carbon and hydrogen recovery rates as a process control index to obtain a heteroatom-removed oil-phase product with carbon and hydrogen elements as main components.

In a hydrogenation process of the prior art, carbon and hydrogen recovery rates are not used as the oil production rate index. Due to the presence of heteroatoms such as oxygen and nitrogen, there is an illusion of high oil production caused by non-carbon-and-hydrogen elements.

According to some embodiments of the present disclosure, the catalyst for removing heteroatoms comprises one or more of Ni/Al₂O₃, Mo/Al₂O₃, and Co/Al₂O₃.

According to some preferred embodiments of the present disclosure, the hydrogenation catalyst comprises one or more of Pt/C, Pt/y-Al₂O₃, Pd/C, Ni—Mo/Al₂O₃, and Co-Mo/Al₂O₃.

According to some embodiments of the present disclosure, a mass ratio between the catalyst for removing heteroatoms and the hydrogenation catalyst is in a range of (10-25): (75-90).

According to specific embodiments of the present disclosure, in the step S4, a hydrogen partial pressure is in a range from 5 to 10 MPa, a hydrogen to oil ratio is in a range from 500 to 800 m³/m³, a temperature is in a range from 275° C. to 400° C., preferably in a range from 260° C. to 325° C., and a superficial flow velocity is in a range from 0.20 to 1 h⁻¹.

According to some embodiments of the present disclosure, the hydrogenated product is subjected to the fractionation process to obtain the kerosene component and the naphtha component, and the kerosene component is a jet biofuel.

Specific limitations are not made to the fractionation process, and common means in the art may be used.

According to preferred embodiments of the present disclosure, the method further comprises a step S6 of performing a hydroisomerisation and hydrocracking process on the kerosene component obtained in step S5. When a proportion of C18-C20 hydrocarbon fuels in the kerosene component is greater than 6%, the kerosene component is subjected to the hydroisomerisation and hydrocracking process.

In one preferred embodiment of the present disclosure, a cracking catalyst can be used in the hydrocracking process. The cracking catalyst can be one or more of Ni—Mo/B₂O₃-Al₂O₃, No—Co/B₂O₃-Al₂O₃, and Ni—Mo/SiO₂—Al₂O₃. In order to further improve low temperature fluidity, a hydroisomerisation process is performed. A hydroisomerisation catalyst can be used in the hydroisomerisation process. The hydroisomerisation catalyst can be one or more of Pt/Al₂O₃—F and Ni—Mo-W/Al₂O₃—F. In the hydroisomerisation and hydrocracking process, a hydrogen partial pressure is in a range from 3 to 12 MPa, a hydrogen to oil ratio is in a range from 500 to 1000 m³/m³, a temperature is in a range from 300° C. to 45° C., preferably in a range from 350° C. to 450° C., and a superficial flow velocity is in a range from 0.25 to 1 h⁻¹.

By performing the heteroatom removing process before the hydrotreating process as well as controlling the process conditions in step S4, the present disclosure realizes hydrogenation after removal of heteroatoms, which can efficiently reduce hydrogen consumption by 1.0% to 5.0% by weight.

In one preferred embodiment of the present disclosure, the method further comprises a step S7 of filtering the water phase obtained in step S3, and returning the filtered water phase as a nutritive salt to the microalgae cultivation pond.

The liquid-phase product in a flash hydrothermal liquefaction obtained by the method of the present disclosure contains elements of carbon, nitrogen, and phosphorus obtained from microalgae. The liquid is filtered and returned to the cultivation pond to provide necessary growth elements for microalgae. The nutrient solution not only enhances the bio-oils yield and avoids discharge of wastewater, but also avoids use of fertilizers for cultivation of biomass so as to reduce energy consumption in a cultivation process.

According to another aspect, the present disclosure provides a use of the jet biofuel prepared by the above method. The jet biofuel prepared by the present disclosure can be used in six types of engines-aircrafts as follows.

The jet biofuel of the present disclosure can be used in single aisle, of which the aircraft types include Airbus A320 series and Boeing 737 series, the engines include CFM56-5B, V2500-A5, and JT8D, and the passenger capacity is in a range from 160 to 180 passengers.

The jet biofuel of the present disclosure can be used in small twin aisle, of which the main aircraft types include Airbus A330 series and Boeing 787-8 series, the engines include PW4000 series, Trent 700 series, and CF6-80 series, and the passenger capacity is in a range from 200 to 250 passengers.

The jet biofuel of the present disclosure can be used in large twin aisle, of which the main aircraft types include Airbus A350 series and Boeing 777 series, the engines include Trent XWB and the passenger capacity is in a range from 290 to 310 passengers.

The jet biofuel of the present disclosure can be used in large quad, of which the main aircraft types include Airbus A380 series and Boeing 747 series, the engines include Trent 900, GP7200, PW JT9D-7R4G2, GE CF6-80C2B1, and RR RB211-524D4, and the passenger capacity is in a range from 550 to 854 passengers.

The jet biofuel of the present disclosure can be used in regional jet, of which the main aircraft types include ERJ145 series, the engines include AE3007, and the passenger capacity is a range from 80 to 100 passengers.

The jet biofuel of the present disclosure can be used in business jet, of which the main aircraft types include Gulfstream G550 and Falcon 7X, the engines include turbofans BR710 and PW307A, and the passenger capacity is in a range from 6 to 10 passengers.

For the engines of the above six types of aircrafts, compared with use of petroleum-based fuels, the use of the jet biofuel of the present disclosure with the mixing ratio being 100% can lead to reductions in emission amounts of per-kilogram of the jet biofuel per kilometer during whole life cycle by 0.494 g, 0.536 g, 0.406 g, 0.492 g, 0.618 g, and 1.86 g, respectively. During whole life cycle, a reduction portion of greenhouse gas emission can reach 50% to 80%. In a perspective of an emission reduction amount, business jet and general aviation aircrafts have maximum potential in emission reduction.

According to another aspect, the present disclosure further provides a low-carbon jet biofuel system based on a whole life cycle. The system comprises a selection module, which is configured to screen and obtain microalgae coupling with carbon spectrum characteristics of jet fuels; a cultivation module, which is configured to cultivate the microalgae to obtain oleaginous microalgae having a strong carbon dioxide fixing ability and a high productivity of fatty acids; a raw material module, which is configured to extract lipid from the oleaginous microalgae to obtain biocrude containing lipid; a preparation module, which is configured to sequentially perform a heteroatom removing process, a hydrotreating process, and a distillation process to obtain a jet biofuel; and an application module, which is configured to apply the jet biofuel to an aircraft engine.

According to some embodiments of the present disclosure, content of C8-C16 hydrocarbon compounds in the jet fuel is in a range from 94.5% to 98%, content of C6-C8hydrocarbon compounds is less than 2%, and content of C17-C20 hydrocarbon compounds is less than 3.5%.

According to some specific embodiments, the hydrocarbon compounds include, but are not limited to, alkanes, cycloalkanes, aromatic hydrocarbons, and olefins.

Microalgae can transform CO₂ into high energy density glycerides. Different microalgae have fatty acid glycerides with different carbon chain lengths. Microalgae which have a fatty acid carbon chain length more relatively similar to carbon spectrum characteristics of jet fuels are selected for breeding and cultivating, so that unicellular or multicellular microalgae which have components similar to carbon spectrum characteristics of jet fuels can be obtained.

According to preferred embodiments of the present disclosure, in the microalgae coupling with carbon spectrum characteristics of jet fuels, fatty acids with a carbon chain length of less than 16 make up 80 to 90% of total fatty acids of the microalgae.

According to preferred embodiments of the present disclosure, strains of the oleaginous microalgae obtained by screening are cultivated preferably in a raceway pond reactor and/or a flat panel photobioreactor.

During cultivation, microalgae which have a high growth rate and a strong lipid accumulation capacity are selected to obtain the oleaginous microalgae, which have a strong carbon dioxide fixing ability and a high yield of fatty acids.

According to preferred embodiments of the present disclosure, the oleaginous microalgae have a carbon dioxide fixing ability in a range from 35 to 60 g/m²·d and a productivity of fatty acid in a range from 5 to 20 g/m²·d.

According to preferred embodiments of the present disclosure, the oleaginous microalgae have oil mass content in a range from 20% to 65% and a growth rate in a range from 20 to 30 g/m²·d.

The oleaginous microalgae of the present disclosure not only have a characteristic of coupling with carbon spectrum characteristics of jet fuels required for aircrafts, but also have characteristics, such as high carbon dioxide fixing ability, high fatty acid yield, and fast growth rate. The oleaginous microalgae with the above characteristics have a relatively high yield of oil, and more oil per unit time can be produced. That is, time required for producing unit oil is shorter, and energy consumption is less. Therefore, low energy consumption of the microalgae during a cultivation stage is guaranteed, and thus energy consumption and greenhouse gas emissions during an entire jet fuel preparation process are also reduced. Hence, the purpose of low-carbon is achieved.

According to some specific embodiments of the present disclosure, the unicellular microalgae comprise nannochloropsis, eustigmatophyceae, and scenedesmus. The nannochloropsis have lipid content in a range from 20% to 44% and a growth rate in a range from 20 to 30 g/m²·d. Carbon spectrums of the nannochloropsis are characterized by C14, C16, and C18. In the nannochloropsis, the fatty acids containing 16 carbons are the main fatty acids, and the fatty acids containing 18 and 14 carbons are in the second rich. The multicellular algae are filamentous algae, which have lipid content in a range from 30% to 65% and a growth rate in a range from 20 to 30 g/m²·d. Carbon spectrums of the filamentous algae are characterized by C14, C16, and C18. In the filamentous algae, the fatty acids containing 16 carbons are the main fatty acids, and the fatty acids containing 18 and 14 carbons are in the second place. The above oleaginous microalgae grow fast, are suitable for stable scale cultivation, and are rich in biologically active components. Besides, the multicellular algae are easy to harvest.

According to preferred embodiments of the present disclosure, for unicellular microalgae, after cultivation is finished, preferably, pH adjustment in-situ flocculation harvest technology is used first, and then the oleaginous microalgae are harvested by a using a centrifugation dewatering process. Compared with a method of adding flocculant or single using centrifugation, energy consumption can be reduced by using the pH adjustment in-situ flocculation harvest technology and recycling of a culture solution is ensured.

According to preferred embodiments of the present disclosure, the raw material module comprises extracting lipid from the oleaginous microalgae by using the flash hydrothermal method to obtain an oil phase, a water phase, a gas phase, and algal residues, wherein the lipid enters the oil phase to form the biocrude.

The term “flash hydrothermal reaction” refers to a process in which biomass is transferred into bio-oils after a series of physical and chemical reactions in a water solution at high temperature and high pressure. Since microalgae grow in a water environment, the harvested microalgae contain a large quantity of water. For preparation of biofuels using an existing pyrolysis process, the microalgae obtained need to be pre-dried, which would cause great energy consumption. For a solvent extraction process, a large number of solvents are needed, and volatilization of the solvents would also result in energy consumption. The present disclosure has a significant energy saving advantage on dealing with microalgae containing water and has a significant energy saving effect by using the flash hydrothermal method, and an energy saving effect is significant. Moreover, the yield increased by 5% to 10%.

In one preferred embodiment of the present disclosure, the flash hydrothermal method is performed preferably in a nitrogen atmosphere at a temperature in a range from 250° C. to 300° C. with a retention time in a range from 1 s to 5 s. A relatively great yield of biocrude would be obtained with such a relatively high temperature and relatively short retention time, and a lipid recovery rate can be more than 98%.

According to some embodiments of the present disclosure, the raw material module specifically comprises treating the oleaginous microalgae in a hydrothermal reactor by using the flash hydrothermal method, followed by obtaining the oil phase, the water phase, the gas phase, and the algal residues by using a flash fractionation process, wherein the lipid enters the oil phase to form the biocrude.

The temperature, pH value, and retention time of the flash hydrothermal method can be adjusted according to mass ratios of oil, protein, and carbohydrate in the microalgae. The nitrogen content in the oil phase may be reduced by 1% to 5% so as to reduce hydrogen consumption in the hydrotreating process.

According to some embodiments of the present disclosure, the preparation module comprises removing heteroatoms from the biocrude in presence of a catalyst for removing heteroatoms to obtain a heteroatom-removed product, and then hydrotreating the heteroatom-removed product in presence of a hydrogenation catalyst to obtain a hydrogenated product.

According to preferred embodiments of the present disclosure, in the heteroatom removing process, oxygen, nitrogen and metals are removed with carbon and hydrogen recovery rates as a process control index to obtain a heteroatom-removed oil-phase product with carbon and hydrogen elements as main components.

In a hydrogenation process of the prior art, carbon and hydrogen recovery rates are not used as the oil production rate index. Due to the presence of heteroatoms such as oxygen and nitrogen, there is an illusion of high oil production caused by non-carbon-and-hydrogen elements.

According to some embodiments of the present disclosure, the catalyst for removing heteroatoms comprises one or more of Ni/Al₂O₃, Mo/Al₂O₃, and Co/Al₂O₃.

According to some preferred embodiments of the present disclosure, the hydrogenation catalyst comprises one or more of Pt/C, Pt/γ—Al₂O₃, Pd/C, Ni—Mo/Al₂O₃, and Co—Mo/Al₂O₃.

According to some embodiments of the present disclosure, a mass ratio between the catalyst for removing heteroatoms and the hydrogenation catalyst is in a range of (10-25):(75-90).

According to specific embodiments of the present disclosure, in the step S4, a hydrogen partial pressure is in a range from 5 to 10 MPa, a hydrogen to oil ratio is in a range from 500 to 800 m³/m³, a temperature is in a range from 275° C. to 400° C., preferably in a range from 260° C. to 325° C., and a superficial flow velocity is in a range from 0.20 to 1 h⁻¹.

According to some embodiments of the present disclosure, the hydrogenated product is subjected to the fractionation process to obtain the kerosene component and the naphtha component, and the kerosene component is a jet biofuel.

Specific limitations are not made to the fractionation process, and common means in the art may be used.

According to preferred embodiments of the present disclosure, the method further comprises a step S6 of performing a hydroisomerisation and hydrocracking process on the kerosene component obtained in step S5. When a proportion of C18-C20 hydrocarbon fuels in the kerosene component is greater than 6%, the kerosene component is subjected to the hydroisomerisation and hydrocracking process.

In one preferred embodiments of the present disclosure, a cracking catalyst can be used in the hydrocracking process. The cracking catalyst can be one or more of Ni—Mo/B₂O₃—Al₂O₃, No—Co/B₂O₃-Al₂O₃, and Ni—Mo/SiO₂—Al₂O₃. In order to further improve low temperature fluidity, a hydroisomerisation process is performed. A hydroisomerisation catalyst can be used in the hydroisomerisation process. The hydroisomerisation catalyst can be one or more of Pt/Al₂O₃—F and Ni—Mo—W/Al₂O₃—F. In the hydroisomerisation and hydrocracking process, a hydrogen partial pressure is in a range from 3 to 12 MPa, a hydrogen to oil ratio is in a range from 500 to 1000 m³/m³, a temperature is in a range from 300° C. to 450° C., preferably in a range from 350° C. to 450° C., and a superficial flow velocity is in a range from 0.25 to 1 ⁻¹.

By performing the heteroatom removing process before the hydrotreating process as well as controlling process conditions in step S4, the present disclosure realizes hydrogenation after removal of heteroatoms, which can efficiently reduce hydrogen consumption by 1.0% to 5.0% by weight.

In one preferred embodiment of the present disclosure, the water phase obtained in the raw material module is filtered, and then returned to the microalgae cultivation pond as a nutritive salt.

The liquid-phase product in a flash hydrothermal liquefaction obtained by the method of the present disclosure contains elements of carbon, nitrogen, and phosphorus obtained from microalgae. The waste liquid is filtered and returned to the cultivation pond to provide necessary growth elements for microalgae. The nutrient solution not only enhances a yield of biocrude and avoids discharge of wastewater, but also avoids use of fertilizers for cultivation of biomass so as to reduce energy consumption in a cultivation process.

According to some embodiments of the present disclosure, the application module can be applied to, but are not limited to, engines for six types of engines -aircrafts as follows.

The jet biofuel of the present disclosure can be used in single aisle, of which the aircraft types include Airbus A320 series and Boeing 737 series, the engines include CFM56-5B, V2500-A5, and JT8D and the passenger capacity is in a range from 160 to 180 passengers.

The jet biofuel of the present disclosure can be used in small twin aisle, of which the man aircraft types include Airbus A330 series and Boeing 787-8 series, the engines include Pw4000 series, Trent 700 series, and CF6-80 series, and the passenger capacity is in a range from 200 to 250 passengers.

The jet biofuel of the present disclosure can be used in large twin aisle, of which the main aircraft types include Airbus A350 series and Boeing 777 series, the engines include Trent XWB, and the passenger capacity is in a range from 290 to 310 passengers.

The jet biofuel of the present disclosure can be used in large quad, of which the main aircraft types include Airbus A380 series and Boeing 747 series, the engines include Trent 900, GP7200, PW JT9D-7R4G2, GE CF6-80C2B1, and RR RB211-524D4, and the passenger capacity is in a range from 550 to 854 passengers.

The jet biofuel of the present disclosure can be used in regional jet, of which the main types include ERJ145 series, the engines include AE3007, and the passenger capacity is in a range from 80 to 100 passengers.

The jet biofuel of the present disclosure can be used in business jet, of which the main types include Gulfstream G550 and Falcon 7X, the engines include turbofans BR710 and PW307A, and the passenger capacity is in a range from 6 to 10 passengers.

Based on consumption and emission characteristics of jet biofuel in processes of taking-off, climbing, approaching, taxiing, and cruising, emissions include three most important greenhouse gases (CO₂, CH₄, and N₂O) and five pollutant emissions (VOC, CO, NOx, PM10, PM2.5, and SOX). According to an average load and a maximum range, for the engines of the above six types of aircrafts, the use of the jet biofuel of the present disclosure with the mixing ratio being 100% can lead to reductions in emission amounts of per-kilogram of the jet biofuel in per kilometer during whole life cycle are by 0.494 g, 0.536 g, 0.406 g, 0.492 g, 0.618 g, and 1.86 g, respectively. During whole life cycle, a reduction portion of greenhouse gas emission can reach 50% to 80%. In a perspective of an emission reduction amount, corporate aircrafts and general aviation aircrafts have minimum potential in emission reduction.

The present disclosure has beneficial effects as follows.

1. In order to ensure performance and safety of aeroengines and aircrafts, requirements of jet fuels such as vaporability, fluidity, combustibility, cleanability and safety are attributed to group composition and carbon spectrum distribution of hydrocarbon fuels, so that direct feedback and optimization of raw materials and preparation processes are realized.

2. The present disclosure focuses on process optimization from a perspective of whole life cycle, and is based on a requirement of adapting to carbon spectrums of engines in a fuel application stage. Selection and cultivation of raw materials is optimized. Carbon distribution of oil is similar to that of jet fuels and thus energy consumption in the preparation stage is effectively reduced.

3. In a microalgae cultivation process, use of nitrogen nutrients is an energy consuming unit, as a result of the relatively high energy consumption in the preparation process. The present disclosure utilizes flash by-product in hydrothermal liquid phase to provide nitrogen in the microalgae cultivation process, thereby reducing carbon emissions caused by use of nutrient salts in a cultivation stage.

4. The present disclosure takes a hydrocarbon yield as a process control index in a fuel preparation stage, and effectively improves the yield and quality of oil products through joint control of heating rate, reaction time, reaction temperature, and cooling time and manner.

5. Content of nitrogen and oxygen in the crude oils is reduced by optimization of the flash hydrothermal liquefaction process. Moreover, hydrogen consumption is reduced and the yield of aviation kerosene is increased by two stages of hydrogenation process.

6. In a perspective of raw material selection, microalgae which are similar to the carbon spectrum characteristics of aviation kerosene are selected in the present disclosure. Therefore, the prepared jet fuel is more conducive to aviation use, and a mixing ratio between the prepared jet fuel and aviation kerosene can be increased by 50% to 100%.

7. The method of the present disclosure has an energy-saving and low-carbon effect. A proportion of greenhouse gas emission reduction over whole life cycle can reach 50% to 80%, and energy consumption can be reduced by 20% to 50%.

### BRIEF DESCRIPTION OF THE DRAWINGS

The accompanying drawings provide further understandings of the present disclosure and constitute one part of the description. The drawings are used for interpreting the present disclosure together with the embodiments, not for limiting the present disclosure. In the drawings:

FIG. 1 is a flowchart showing a method for preparing a low-carbon jet biofuel based on whole life cycle assessment according to the present disclosure.

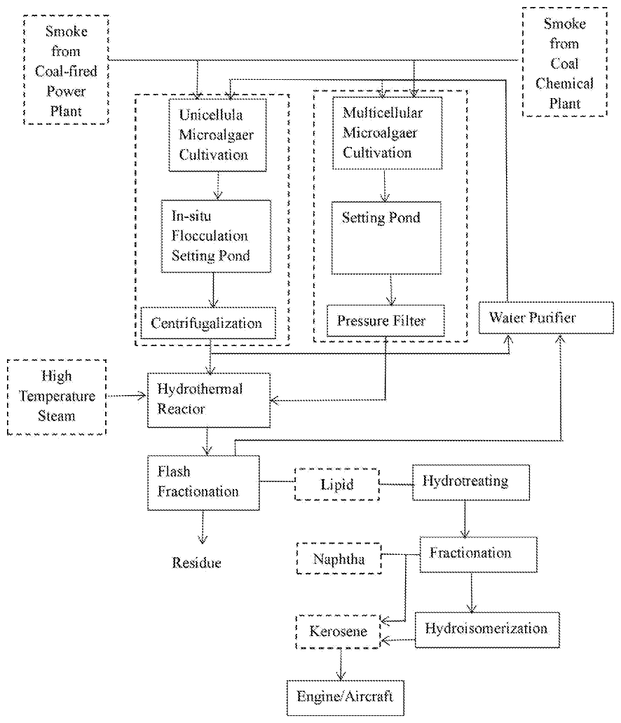

### DETAILED DESCRIPTION OF THE EMBODIMENTS

The technical solutions of the present disclosure will be further explained below with reference to the embodiments.

### EXAMPLE 1

(1) Selection of Microalgae was as follows. Nannochloropsis algae with oil content in a range from 20% 40%, a growth rate in a range from 20 to 30 g/m²·d, and carbon spectrums of C14, C16, and C18 were selected.

(2) Carbon dioxide (CO₂) from a coal-fired power plant was purified or directly fed to a microalgae cultivation pond. An amount of CO₂ was adjusted through control of pH value, and the pH value was controlled in a range from 5.5 to 7.8. In-situ harvest of the Nannochloropsis algae is realized through control of pH value. The pH value for in-situ harvest was either in a range from 4.0 to 4.8 under acidic conditions, or in a range from 9.5 to 10.5 under alkaline conditions. A concentration for in-situ harvest could be in a range from 20 to 30 g/L. After centrifugal dewatering, the concentration was in a range from 150 to 250 g/L. After filtering and disinfecting, water collected by harvest and dewatering was returned to the cultivation pond.

(3) Microalgae pulp was delivered into a hydrothermal reactor which had been heated to a preset temperature by means of a high-pressure pump. The temperature of the reactor was controlled in a range from 270° C. to 300° C. Nitrogen was fed to the reactor with a retention time in a range from 5 to 30 min to keep an inert atmosphere in the reactor. Since it takes time for cell wall rupture of the Nannochloropsis, the heating rate was less than 100° C./min. Materials from an outlet of the hydrothermal reactor were fed to a multiphase separator, which was provided with a filter therein. Gas was discharged from an upper part of the multiphase separator. Solid residues were precipitated at a bottom part of the multiphase separator and then discharged at the bottom part thereof. A liquid portion overflowed from the filter provided at the upper part of the multiphase separator, so that a liquid bio-oil and water were obtained. An oil phase and a water phase were separated to obtain a biocrude. After purification and disinfection, the water phase was returned to the cultivation pond to provide nitrogen sources.

(4) The biocrude was subjected to a heteroatom removal process and a hydrotreating process first. In a hydrogenation reactor, a catalyst for removing heteroatoms was provided at an upper layer, and a hydrogenation catalyst was provided at a lower layer. The catalyst for removing heteroatoms was Ni/Al₂O₃, a hydrogen partial pressure was in a range from 8 to 10 MPa, a hydrogen to oil ratio was in a range from 400 to 800 m³/m³, a temperature was in a range from 260° C. to 300° C., and a velocity of flow was in a range from 0.25 to 2 ⁻¹. The hydrogenation catalyst was Ni—Mo/Al₂O₃, a hydrogen partial pressure was in a range from 5 to 10 MPa, a hydrogen to oil ratio was in a range from 600 to 1000 m³/m³, a temperature was in a range from 280° C. to 400° C., and a superficial flow velocity was in a range from 0.25 to 2 h⁻¹. After the heteroatom removing and the hydrotreating process, a hydrocarbon distillate could be obtained. Next, a fractionation process was performed. A light distillate carbon number distribution was in a range from C8 to C16, and a fractionation temperature distribution was in a range from 160° C. to 280° C. A high distillate carbon number distribution was in a range from C17 to C32, and a fractionation temperature distribution was in a range from 160° C. to 350° C. Besides, there was 15% to 15% of naphtha fuel having carbon number distribution less than C7.

(5) The high hydrocarbon distillate was hydrocracked to obtain a low distillate hydrocarbon fuel. The low distillate hydrocarbon fuel obtained by hydrocracking and a low-distillate hydrocarbon fuel obtained by hydrotreating were performed with a hydroisomerization process to obtain a jet fuel that met performance requirements. A hydrocracking catalyst was Ni—Mo/SiO₂—Al₂O₃, a hydroisomerization catalyst was Pt/Al₂O₃—F, and a hydrogen partial pressure was in a range from 3 to 15 MPa. A hydrogen to oil ratio was in a range from 1000 to 1500 m³/m³, a temperature was in a range from 400° C. to 450° C., and a superficial flow velocity was in a range from 0.25 to 2 h⁻¹.

(6) The obtained aviation kerosene had a heat value in a range from 43.0 to 43.7 MJ/kg, a density in a range from 780 to 810 kg/m3, a total acid number in a range from 0.002-0.005 mgKOH/g, total nitrogen content in a range from 1 to 2 ppm, and total sulphur content in a range from 0.04% to 0.08%, and could be used in six types of airplanes and engines thereof. Carbon emissions over whole life cycle were reduced by more than 50%. Such biofuel could be fully used in business jet, and emissions per kilogram of the biofuel per kilometer during whole life cycle could be reduced by 1.5 g.

### EXAMPLE 2

(1) Selection of microalgae was as follows. Multicellular Tribonema with oil content in a range from 30% to 65%, a growth rate in a range from 20 to 30 g/m²·d, and carbon spectrums of C14, C16, and C18 were selected.

(2) Carbon dioxide (CO₂) from a coal-fired power plant was purified or directly fed to a Tribonema cultivation pond. An amount of CO₂ was adjusted through control of pH value, and the pH value was controlled in a range from 5.5 to 7.8. Tribonema were large, and thus could be harvested directly. Therefore, a dewatering process was no longer needed. Tribonema were rapidly filtered by using nylon bolting cloth of 200 to 400 mesh. After squeezing and dewatering, Tribonema had a concentration of 350 g/L. After filtering and disinfecting, water collected by harvest and dewatering was returned to the cultivation pond.

(3) Microalgae pulp was delivered into a hydrothermal reactor which had been heated to a preset temperature by means of a high-pressure pump. The temperature of the reactor was controlled in a range from 270° C. to 300° C. Nitrogen was fed to the reactor with a retention time in a range from 5 to 30 min to keep an inert atmosphere in the reactor. Since it takes time for cell wall rupture of the Nannochloropsis, a heating rate was less than 100° C/min. Materials from an outlet of the hydrothermal reactor were fed to a multiphase separator, which was provided with a filter therein. Gas was discharged from an upper part of the multiphase separator. Solid residues were precipitated at a bottom part of the multiphase separator and then discharged at the bottom part thereof. A liquid portion overflowed from the filter provided at the upper part of the multiphase separator, so that a liquid bio-oil and water were obtained. An oil phase and a water phase were separated to obtain a biocrude. After purification and disinfection, the water phase was returned to the cultivation pond to provide nitrogen sources.

(4) The biocrude was subjected to a heteroatom removal process and a hydrogenation process first. In a hydrogenation reactor, a catalyst for removing heteroatoms was provided at an upper layer, and a hydrogenation catalyst was provided at a lower layer. The catalyst for removing heteroatoms was Ni/Al₂O₃, a hydrogen partial pressure was in a range from 8 to 10 MPa, a hydrogen to oil ratio was in a range from 400 to 800 m³/m³, a temperature was in a range from 260° C. to 300° C., and a superficial flow velocity was in a range from 0.25 to 2 h⁻¹. The hydrogenation catalyst was Ni—Mo/Al₂O₃, a hydrogen partial pressure was in a range from 5 to 10 MPa, a hydrogen to oil ratio was in a range from 600 to 1000 m³/m³, a temperature was in a range from 280° C. to 400° C., and a superficial flow velocity was in a range from 0.25 to 2 h⁻¹. After the heteroatom removing process and the hydrotreating process, a hydrocarbon distillate could be obtained. Next, a fractionation process was performed. A light distillate carbon number distribution was in a range from C8 to C16, and a high distillate carbon number distribution was in a range from C17 to C32. Besides, there was 5% to 15% of a naphtha fuel having carbon number distribution less than C7.

(5) The high hydrocarbon distillate was hydrocracked to obtain a low-distillate hydrocarbon fuel. The low distillate hydrocarbon fuel obtained by hydrocracking and a low-distillate hydrocarbon fuel obtained by hydrotreating were performed with a hydroisomerization process to obtain a jet fuel that met performance requirements. A hydrocracking catalyst was Ni—Mo—W/SiO₂—Al₂O₃. A hydroisomerization catalyst was Mo—W—Ni/Al₂O₃—F. A hydrogen partial pressure was in a range from 3 to 15 MPa, a hydrogen to oil ratio was in a range from 1000 to 1500 m³/m³, a temperature was in a range from 400° C. to 450° C., and a superficial flow velocity was in a range from 0.25 to 2 h⁻¹.

(6) The obtained aviation kerosene had a heat value in a range from 43.0 to 43.7 MJ/kg, a density in a range from 780 to 810 kg/m³, a total acid number in a range from 0.002-0.005 mgKOH/g, total nitrogen content in a range from 1 to 2 ppm, and total sulphur content in a range from 0.04% to 0.08%, and could be used in six types of airplanes and engines thereof. Carbon emissions over full life cycle were reduced by more than 60% to 80%. Such biofuel could be fully used in cooperation aircrafts, and emissions per kilogram of the biofuel per kilometer during lifecycle could be reduced by 1.86 g.

It should be noted that the above embodiments are only used for explaining the present disclosure, rather than limiting the present disclosure. The present disclosure has been described with reference to the exemplary embodiments, but it should be understood that words used in the embodiments are explanatory words, rather than definitive words. The present disclosure can be modified within the scope of the claims of the present disclosure according to regulation. Also, amendments can be made to the present disclosure without departing from the scope and spirit of the present disclosure. Although the present disclosure relates to specific methods, materials, and embodiments, it is not intended that the present disclosure be limited to the specific embodiments disclosed here. The present disclosure can be extended to all other methods and applications having same functions.

1. A method for preparing low-carbon jet biofuel based on whole life cycle, the method comprising steps of:
S1, screening and obtaining microalgae coupling with carbon spectrum characteristics of jet fuels;
S2, cultivating the microalgae to obtain oleaginous microalgae, which have a strong carbon dioxide fixing ability and a high productivity of fatty acids;
S3, extracting lipid from the oleaginous microalgae using a flash hydrothermal method to obtain biocrude containing lipid;
S4, subjecting the biocrude to a heteroatom removing process and a hydrotreating process sequentially to obtain a hydrogenated product; and
S5, subjecting the hydrogenated product to a fractionation process to obtain a kerosene component and a naphtha component, wherein the kerosene component is a jet biofuel.

2. The method according to claim 1, wherein the oleaginous microalgae have a carbon dioxide fixing ability in a range from 35 to 60 g/m²·d.

3. The method according to claim 1, wherein the oleaginous microalgae have a yield of fatty acid in a range from 5 to 20 g/m²·d.

4. The method according to claim 1, wherein the oleaginous microalgae have a lipid content in a range from 20% to 65%.

5. The method according to claim 1, wherein the oleaginous microalgae have a growth rate in a range from 20 to 30 g/m²·d.

6. The method according to claim 1, wherein the step 4 comprises steps of:
4A, subjecting the biocrude to a heteroatom removing process in presence of a catalyst for removing heteroatoms to remove heteroatoms from an oil phase and obtain a heteroatom-removed product, and
4B, subjecting the heteroatom-removed product to a hydrotreating process in presence of a hydrogenation catalyst to obtain a hydrogenated product.

7. The method according to claim 6, wherein the catalyst for removing heteroatoms comprises one or more of Ni/Al₂O₃, Mo/Al₂O₃, Co/Al₂O₃, and No—Co/Al₂O₃.

8. The method according to claim 6, wherein the hydrogenation catalyst comprises one or more of Pt/C, Pt/γ—Al₂O₃, Pd/C, Ni—Mo/Al₂O₃, and Co—Mo/Al₂O₃.

9. The method according to claim 1, further comprising a step S6 of performing a hydroisomerisation and hydrocracking process on the kerosene component obtained in step S5.

10. Use of the jet biofuel prepared using the method according to claim 1.

11. A low-carbon jet biofuel system based on whole life cycle, comprising:
a selection module, which is configured to screen and obtain microalgae coupling with carbon spectrum characteristics of jet fuels;
a cultivation module, which is configured to cultivate the microalgae to obtain oleaginous microalgae having a strong carbon dioxide fixing ability and a high productivity of fatty acids;
a raw material module, which is configured to extract lipid from the oleaginous microalgae to obtain biocrude containing lipid;
a preparation module, which is configured to sequentially perform a heteroatom removing process, a hydrotreating process, and a fractionation process to the biocrude to obtain a jet biofuel; and
an application module, which is configured to apply the jet biofuel to an aircraft engine.

12. The system according to claim 11, wherein the oleaginous microalgae have a carbon dioxide fixing ability in a range from 35 to 60 g/m²·d.

13. The system according to claim 12, wherein the oleaginous microalgae have a yield of fatty acid in a range from 5 to 20 g/m²·d.

14. The system according to claim 12, wherein the oleaginous microalgae have a lipid content in a range from 20% to 65%.

15. The system according to claim 12, wherein the oleaginous microalgae have a growth rate in a range from 20 to 30 g/m²·d.

16. The system according to claim 11, wherein the preparation of the jet biofuel comprises steps of:
(1) subjecting the biocrude to a heteroatom removing process in presence of a catalyst for removing heteroatoms to remove heteroatoms from an oil phase and obtain a heteroatom-removed product;
(2) subjecting the heteroatom-removed product to a hydrotreating process in presence of a hydrogenation catalyst to obtain a hydrogenated product; and
(3) subjecting the hydrogenated product to a fractionation process to obtain a kerosene component and a naphtha component, wherein the kerosene component is the jet biofuel; and
(4) optionally, subjecting the kerosene component to a hydroisomerisation and hydrocracking process.

17. The system according to claim 16, wherein the catalyst for removing heteroatoms comprises one or more of Ni/Al₂O₃, Mo/Al₂O₃, Co/Al₂O₃, and No—Co/Al₂O₃.

18. The system according to claim 16, wherein the hydrogenation catalyst comprises one or more of Pt/C, Pt/γ—Al₂O₃, Pd/C, Ni—Mo/Al₂O₃, and Co—Mo/Al₂O₃.

19. The system according to claim 11, wherein the application module can be applied in at least one of a single aisle, a small twin aisle, a twin aisle, a large quad, a v, and a business jet.
